(19) United States
(12) Patent Application Publication
(10) Pub. No.: US 2026/0115495 A1
(43) Pub. Date: Apr. 30, 2026

(54) Therapeutic Device for Applying Medicinal Cloth and Ultrasound Cavitation Treatment

(71) Applicant: Zhijun Peng, Foshan (CN)

(72) Inventor: Zhijun Peng, Foshan (CN)

(21) Appl. No.: 18/988,907

(22) Filed: Dec. 20, 2024

(30) Foreign Application Priority Data
Oct. 30, 2024 (CN) 202411530285.X

(51) Int. Cl. A61N 7/02; A61N 7/00

(52) U.S. Cl. CPC A61N 7/00; A61N 2007/0039

(57) ABSTRACT

A therapeutic device for applying medicinal cloth and ultrasound cavitation treatment, comprising a medicinal cloth infiltrated and adsorbed with a medicinal solution, and an ultrasonic vibration therapy device provided with a strap, wherein the medicinal cloth is arranged on the surface of the ultrasonic vibration therapy device, and a clamping mechanism is also provided on the ultrasonic vibration therapy device, and wherein the ultrasonic vibration therapy device comprises a bottom shell and a shell cover forming a receiving cavity, and an ultrasonic vibration assembly and a rechargeable driving control assembly that are installed within the receiving cavity and electrically connected to each other.

### FIELD OF THE INVENTION

The present invention relates to the field of human body massaging instruments, and in particular to an ultrasound cavitation therapeutic device designed for applying medicinal cloth.

### BACKGROUND OF THE INVENTION

As human society continues to advance, people's daily life and work rhythms have accelerated significantly. Working overtime, staying up late, or maintaining a fixed sitting posture for extended periods have become routine. These habits hinder blood circulation in various parts of the human body, affecting metabolism and consequently health. As a result, sub-health conditions such as soreness, pain, poor mental state, and fatigue in the limbs frequently occur.

On Apr. 7, 2020, the applicant filed a patent application with the China National Intellectual Property Administration under the application No. 202010265165.7, published as CN116672239A, titled “A Bendable and Adjustable Airbag Ultrasonic Massage Healthcare Device.” This previous solution primarily includes a wearable item and an ultrasonic massage head installed on the wearable item. Inside the ultrasonic massage head is an inner cavity accommodating an ultrasonic motor or ultrasonic transducer. The outer surface of the ultrasonic massage head is equipped with a pipeline connector linked to the inner cavity and multiple medication-spraying holes. In use, an external electric medication supply unit, connected to the pipeline connector via a hose and to the ultrasonic massage head via an electrical wire for power, can simultaneously spray medication onto the treated body area while applying high-frequency ultrasonic vibration massage, thereby achieving improved therapeutic and healthcare effects and alleviating soreness and pain in the human body.

However, the above therapeutic apparatus has several drawbacks:

During use, it requires an additional electric medication supply unit connected to the ultrasonic therapy device by hoses and electrical wires. The ultrasonic therapy device depends on the supply unit for control and power, resulting in a cumbersome combination of pipelines and wires. This inconvenience limits users from employing it while working or performing daily household chores, thus hindering its portability and ease of use.

The ultrasonic therapy device and the medication-spraying function share the same enclosed inner cavity, placing the ultrasonic therapy component in a moist environment. This situation can compromise the device's safety and longevity. Moreover, as the inner cavity is relatively closed, medication residues adhering to the interior cannot be easily cleaned, creating potential hygiene blind spots where bacteria may accumulate, adversely affecting users' health and safety.

In view of the above shortcomings, the applicant believes that the previous massaging products still have multiple deficiencies. There is a pressing need for redesign and development to better serve human health.

### SUMMARY OF THE INVENTION

The objective of the present invention is to address the above problems and deficiencies by providing a therapeutic device that employs a medicinal cloth soaked with medication in combination with ultrasonic cavitation therapy. In this therapeutic device, the medicinal cloth is laid flat against the bottom surface of the ultrasonic vibration therapy component. When the device is strapped onto the part of the human body requiring treatment, the medicinal cloth is in direct, uniform contact with that area. When the ultrasonic vibration therapy component operates and generates high-frequency ultrasound vibrations, it not only presses out the medication absorbed in the medicinal cloth and evenly applies it to the treated body area but also uses the high-frequency ultrasonic cavitation effect to stimulate cellular activity, accelerate metabolism, and enhance blood circulation. This process quickly clears and opens the skin's pores, enabling rapid absorption of the medication directly into the affected area and achieving improved therapeutic results. Additionally, the design eliminates hygiene blind spots, is easy to clean and wear, and does not interfere with daily chores or work.

The technical solution of the present invention is implemented as follows:

A therapeutic device employing a medicinal cloth for applying medication combined with ultrasonic cavitation therapy, characterized by comprising: a medicinal cloth soaked with medication, and an ultrasonic vibration therapy device equipped with straps. The medicinal cloth is arranged on the bottom surface of the ultrasonic vibration therapy device. The ultrasonic vibration therapy device also includes a clamping mechanism to securely hold the medicinal cloth in a flattened, taut configuration against its bottom surface. The ultrasonic vibration therapy device comprises a bottom shell and a shell cover assembled together to form a receiving cavity, in which an ultrasonic vibration assembly and a rechargeable driving control assembly are installed and electrically connected.

Furthermore, two clamping mechanisms are provided, each arranged at one end of the bottom shell.

In addition, the clamping mechanism may include a fixed plate arranged on the bottom shell, a clamping plate hinged on the fixed plate, and a spring positioned between the fixed plate and the clamping plate. The restoring force of the spring presses one side of the clamping plate against the fixed plate, forming a clamping structure for the medicinal cloth. An insertion gap is formed between the opposite ends of the clamping plate and the fixed plate, and an end of the medicinal cloth is secured therein.

Beneficial Effects: By laying the medicinal cloth flat against the bottom surface of the ultrasonic vibration therapy device and then strapping the device onto the body part in need of treatment, the medicinal cloth can evenly contact the skin. When operating, the ultrasonic vibration therapy device generates high-frequency ultrasonic vibrations that squeeze out and evenly distribute the absorbed medication from the medicinal cloth onto the treated area. Simultaneously, the high-frequency ultrasonic cavitation effect promotes cellular activity, accelerates metabolism and blood circulation, quickly clears and opens the pores, and ensures rapid absorption of the medication deep into the affected area, thereby achieving enhanced therapeutic efficacy. Additionally, no hygiene blind spots remain, cleaning is convenient and swift, and the device is easy to wear without hindering daily chores or work. To renew the medication, simply replace the medicinal cloth, offering convenience, hygiene, and safety.

### BRIEF DESCRIPTION OF THE DRAWINGS

FIG. 1 is a three-dimensional structural schematic diagram of the present invention;

FIG. 2 is a structural schematic diagram after removing the medicinal cloth from the present invention;

FIG. 3 is a schematic diagram showing the application of the first type of clamping mechanism on the ultrasonic vibration therapy device according to the present invention;

FIG. 4 is a schematic diagram showing the application of the second type of clamping mechanism on the ultrasonic vibration therapy device according to the present invention;

FIG. 5 is a first exploded view of the ultrasonic vibration therapy device of the present invention;

FIG. 6 is a second exploded view of the ultrasonic vibration therapy device of the present invention;

FIG. 7 is a usage schematic diagram of the ultrasonic vibration therapy device of the present invention paired with a wireless charger.

### DETAILED DESCRIPTION OF THE EMBODIMENTS

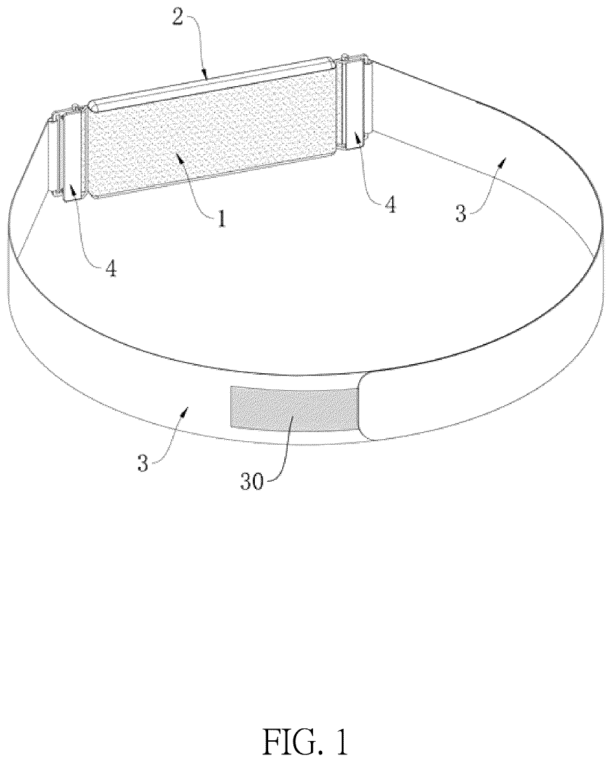

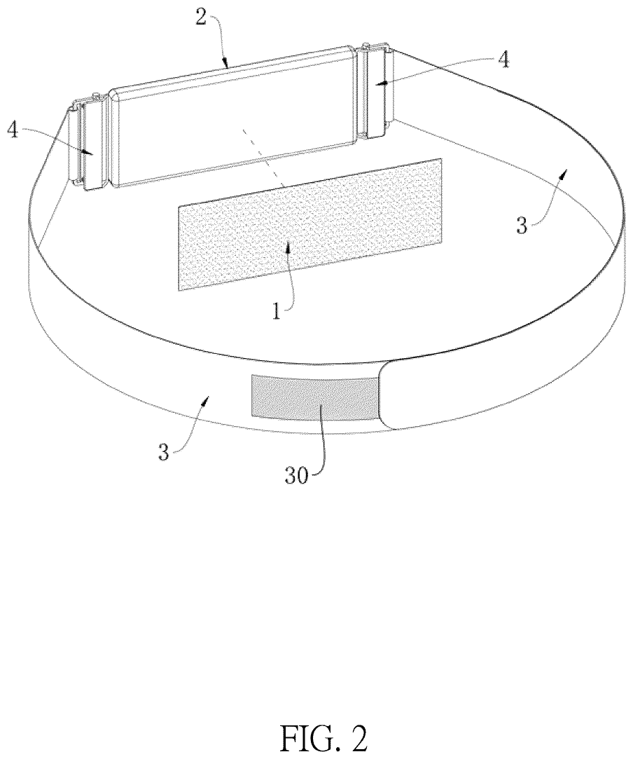

As shown in FIGS. 1 and 2, the therapeutic device employing a medicinal cloth soaked with medication and ultrasonic cavitation therapy described in the present invention comprises a medicinal cloth (1) soaked with medication and an ultrasonic vibration therapy device (2) equipped with a strap (3). The medicinal cloth (1) is placed on the bottom surface of the ultrasonic vibration therapy device (2), and a clamping mechanism (4) is provided on the ultrasonic vibration therapy device (2) to secure the medicinal cloth (1) in a flat and taut state on the bottom surface of the ultrasonic vibration therapy device (2). The ultrasonic vibration therapy device (2) includes a bottom shell (21) and a shell cover (22) assembled together to form a receiving cavity (211). Inside the receiving cavity (211), an ultrasonic vibration assembly (23) and a rechargeable driving control assembly (24) are installed and electrically connected.

When the ultrasonic vibration therapy device (2) operates, it generates high-frequency ultrasonic vibrations. These vibrations cause the medication absorbed in the medicinal cloth (1) to be pressed out and spread evenly onto the treated body area. The medicinal cloth (1) can be a wet wipe or cotton cloth soaked with medication, typically disposable to ensure hygiene and health. The medicinal cloth (1) may be infused with traditional Chinese herbal concoctions or pre-prepared Western medicinal solutions.

As shown in FIG. 1, there are two clamping mechanisms (4), each arranged at one end of the bottom shell (21). These two clamping mechanisms (4) secure both ends of the medicinal cloth (1), ensuring that the cloth (1) remains flat against the bottom surface of the ultrasonic vibration therapy device (2) without slipping or wrinkling, thereby maintaining optimal skin contact.

In practical implementations, the clamping mechanism (4) can take various forms, as long as it can stably secure the medicinal cloth (1) to the bottom surface of the ultrasonic vibration therapy device (2). The present invention provides two specific reference solutions.

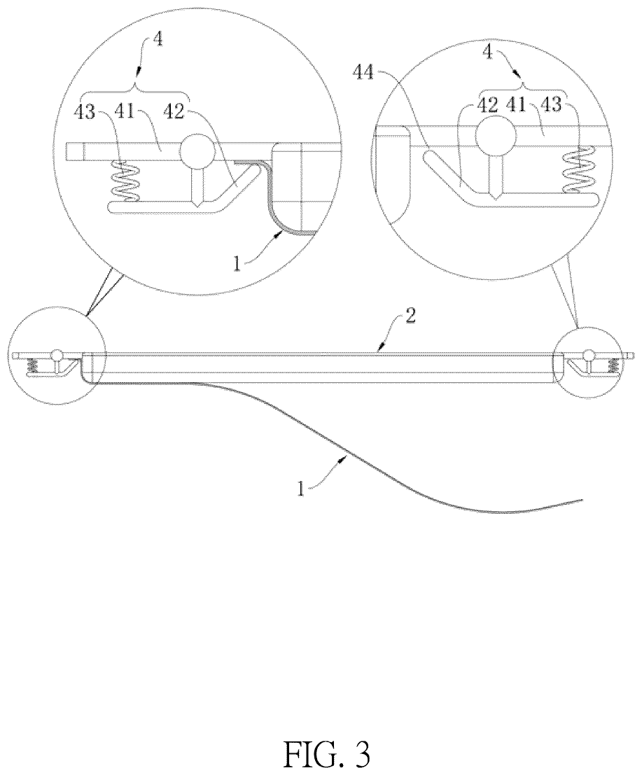

First solution as shown in FIG. 3: The clamping mechanism (4) includes a fixed plate (41) arranged on the bottom shell (21), a clamping plate (42) hinged to the fixed plate (41), and a spring (43) positioned between the fixed plate (41) and the clamping plate (42). The spring's (43) restoring force presses one side of the clamping plate (42) tightly against the fixed plate (41), forming a clamping structure (44) for the medicinal cloth. The end of the medicinal cloth (1) is then secured within this clamping structure (44). In use, simply press down the outer edge of the clamping plate (42) to lift its inner edge and open the clamping structure (44). Insert the end of the medicinal cloth (1) and release pressure on the clamping plate (42), allowing the spring's (43) restoring force to close the clamping structure (44) and firmly hold the medicinal cloth (1).

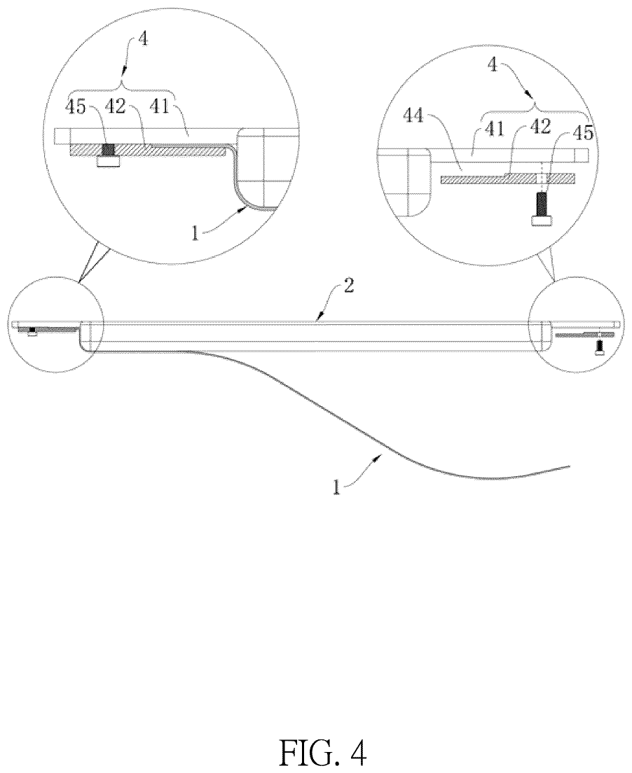

Second solution as shown in FIG. 4: The clamping mechanism (4) includes a fixed plate (41) on the bottom shell (21), a clamping plate (42), and a screw (45). The clamping plate (42) is connected to the fixed plate (41) by the screw (45) to form a clamping structure (44) that holds the end of the medicinal cloth (1). In use, rotate the screw (45) counterclockwise to loosen and open the clamping structure (44). Insert the end of the medicinal cloth (1) into the opened clamping structure (44). Then rotate the screw (45) clockwise to move the clamping plate (42) back, closing the clamping structure (44) and firmly securing the medicinal cloth (1).

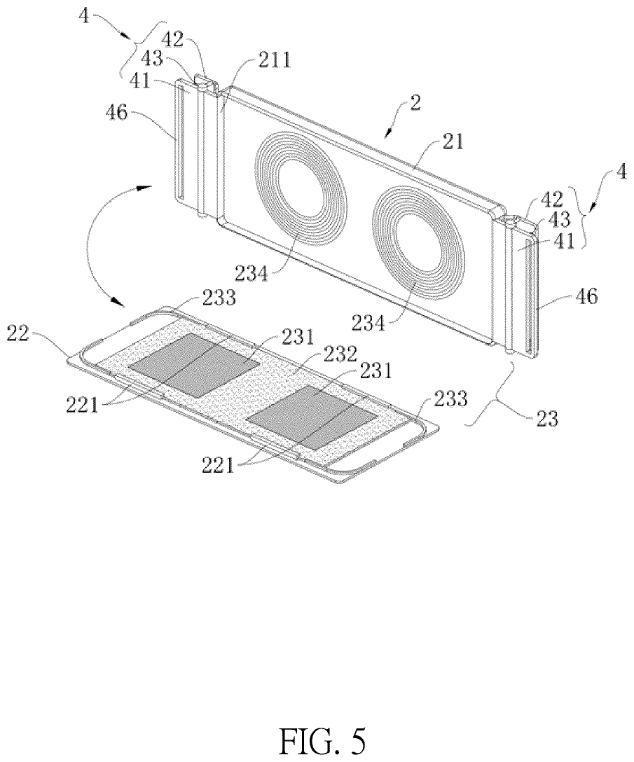

As shown in FIGS. 2 and 5, a protruding ring portion (46) is provided on the fixed plate (41). One end of the strap (3) is tied to this protruding ring portion (46), while the other end of the strap (3) can be formed with a fastening section. Commonly, a hook-and-loop fastener (Velcro) (30) is attached at the fastening end of the strap (3) to facilitate easy securing by the user. Using the strap (3), the ultrasonic vibration therapy device (2) can be securely worn on a human body part for therapy, freeing the user's hands. Alternatively, a simple tying cord can be attached to the protruding ring portion (46) as the strap (3).

As shown in FIG. 5, the ultrasonic vibration assembly (23) comprises a sliding block (232) carrying a permanent magnet (231), elastic tabs (233) installed at both ends of the sliding block (232) to provide buffering and rebound forces, and a driving coil (234) that interacts with the permanent magnet (231) to generate a magnetic drive. The driving coil (234) is electrically connected to the rechargeable driving control assembly (24). The elastic tabs (233) ensure that when the driving coil (234) moves the permanent magnet (231) and sliding block (232), they can return to their initial positions, thereby enabling reciprocating motion. Specifically, the sliding block (232) is movably arranged on the shell cover (22), which is further provided with limiting protrusions (221) to prevent the sliding block (232) from detaching.

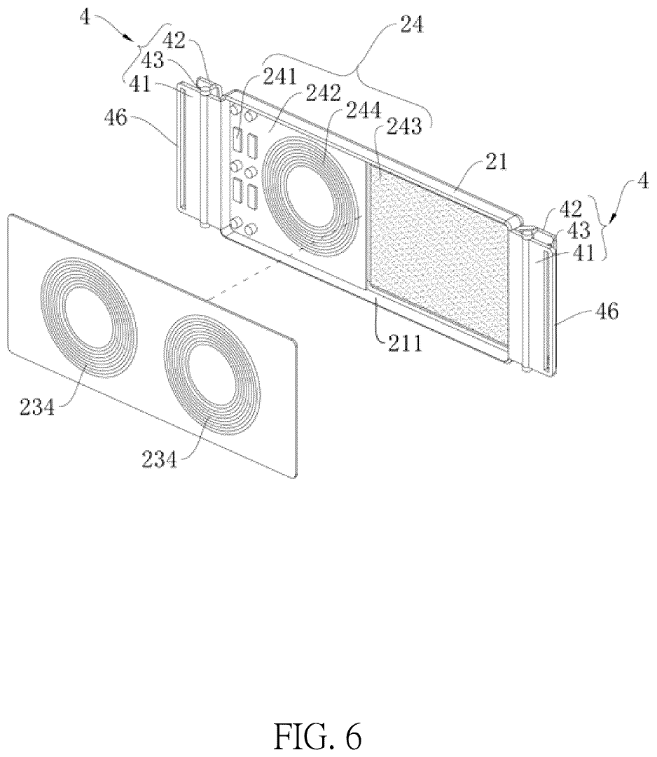

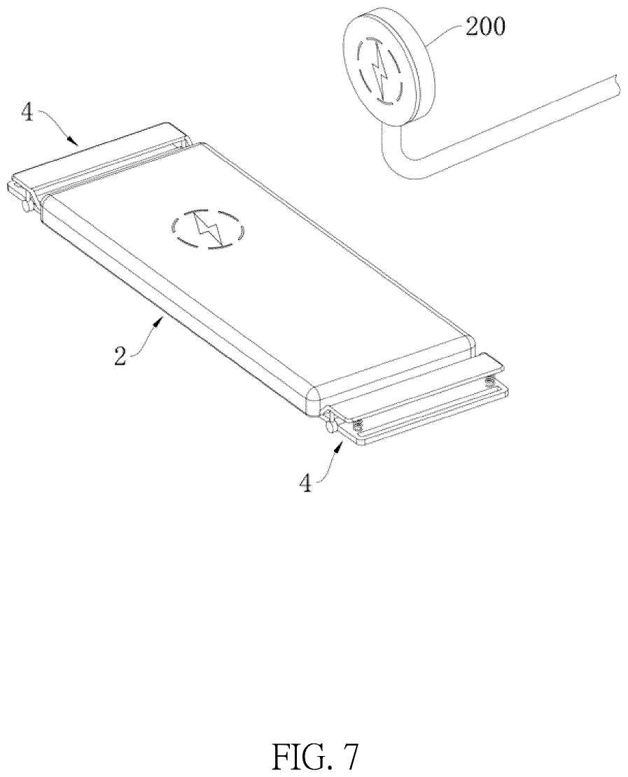

As shown in FIG. 6, the rechargeable driving control assembly (24) comprises a circuit board (242) with a wireless communication module (241), a battery (243) electrically connected to the circuit board (242), and a wireless charging coil (244). The driving coil (234) is electrically connected to the circuit board (242) to drive the ultrasonic vibration assembly (23). By providing the wireless charging coil (244), the ultrasonic vibration therapy device (2) gains wireless charging capabilities, making it more convenient to use. As shown in FIG. 7, the ultrasonic vibration therapy device (2) can be placed over the transmitter end (200) of a wireless charger for wireless charging. With the wireless communication module (241), the device can also connect to the Internet, allowing a computer, smartphone, or other networked device to control the ultrasonic vibration therapy device (2). The wireless communication module (241) may be a Bluetooth module or a Wi-Fi module.

Principle of Operation: The battery (243) supplies current to the driving coil (234) through wires. When current passes through the driving coil (234), a magnetic field is generated, driving the permanent magnet (231) and sliding block (232) inside the receiving cavity (211) to move back and forth at ultra-high frequency, thereby producing ultra-high frequency vibrations. These vibrations propagate through air or solid mediums, forming ultrasonic waves.

The high-frequency ultrasonic vibration mechanism composed of a sliding block with a permanent magnet, elastic tabs, and a driving coil can also be replaced with an ultrasonic transducer or an ultrasonic motor. The clamping mechanism (4) can adopt forms other than those described, as long as it can easily clamp and stabilize the medicinal cloth on the ultrasonic vibration therapy device (2). Furthermore, the shape of the ultrasonic vibration therapy device (2) can be arbitrarily designed to fit various body parts for better adherence during therapy. The illustrated embodiment is merely an example, not the final implementation of the invention.

I claim:

1. A therapeutic device for applying medicinal cloth and ultrasound cavitation treatment, comprising:
a medicinal cloth (1) infiltrated and adsorbed with a medicinal solution and an ultrasonic vibration therapy device (2) provided with a strap (3),
wherein the medicinal cloth (1) is arranged on a bottom surface of the ultrasonic vibration therapy device (2), and a clamping mechanism (4) is provided on the ultrasonic vibration therapy device (2) to clamp and secure the medicinal cloth (1), thereby laying the medicinal cloth (1) flat and holding the medicinal cloth (1) taut against the bottom surface of the ultrasonic vibration therapy device (2),
and wherein the ultrasonic vibration therapy device (2) comprises a bottom shell (21) and a shell cover (22) assembled together to form a receiving cavity (211), and an ultrasonic vibration assembly (23) as well as a rechargeable driving control assembly (24) that are installed within the receiving cavity (211) and electrically connected to each other.

2. The therapeutic device for applying medicinal cloth and ultrasound cavitation treatment according to claim 1, wherein two of the clamping mechanisms (4) are provided, each arranged at one end of the bottom shell (21).

3. The therapeutic device for applying medicinal cloth and ultrasound cavitation treatment according to claim 2, wherein the clamping mechanism (4) comprises a fixed plate (41) arranged on the bottom shell (21), a clamping plate (42) hinged to the fixed plate (41), and a spring (43) positioned between the fixed plate (41) and the clamping plate (42), and the restoring force of the spring (43) presses one side of the clamping plate (42) against the fixed plate (41) forming a clamping structure (44) in which an end of the medicinal cloth (1) is secured.

4. The therapeutic device for applying medicinal cloth and ultrasound cavitation treatment according to claim 3, wherein the fixed plate (41) is further provided with a protruding ring portion (46), one end of the strap (3) is tied to the protruding ring portion (46), and the other end of the strap (3) forms a fastening end.

5. The therapeutic device for applying medicinal cloth and ultrasound cavitation treatment according to claim 2, wherein the clamping mechanism (4) comprises a fixed plate (41) arranged on the bottom shell (21), a clamping plate (42), and a screw (45), and the clamping plate (42) is connected to the fixed plate (41) by means of the screw (45) forming a clamping structure (44) in which an end of the medicinal cloth (1) is secured.

6. The therapeutic device for applying medicinal cloth and ultrasound cavitation treatment according to claim 5, wherein the fixed plate (41) is further provided with a protruding ring portion (46), one end of the strap (3) is tied to the protruding ring portion (46), and the other end of the strap (3) forms a fastening end.

7. The therapeutic device for applying medicinal cloth and ultrasound cavitation treatment according to claim 1, wherein the ultrasonic vibration assembly (23) comprises a sliding block (232) having a permanent magnet (231), elastic tabs (233) arranged at both ends of the sliding block (232) to provide buffering and rebound forces, and a driving coil (234) that cooperates with the permanent magnet (231) to produce a magnetic driving effect, and the driving coil (234) is electrically connected to the rechargeable driving control assembly (24).

8. The therapeutic device for applying medicinal cloth and ultrasound cavitation treatment according to claim 7, wherein the rechargeable driving control assembly (24) comprises a circuit board (242) provided with a wireless communication module (241), a battery (243) electrically connected to the circuit board (242), and a wireless charging coil (244), and the driving coil (234) is electrically connected to the circuit board (242) to drive the ultrasonic vibration assembly (23).
